(19) United States
(12) Patent Application Publication
(10) Pub. No.: US 2011/0015612 A1
(43) Pub. Date: Jan. 20, 2011

(54) IMPLANTABLE DEVICES FOR TREATMENT OF SINUSITIS

(75) Inventors: Benjamin Arcand, Minneapolis, MN (US); Joseph E. Hale, Maplewood, MN (US); Nikhil Murdeshwar, Maple Grove, MN (US); Bryan Rolfes, St. Paul, MN (US)

Correspondence Address: SCHWEGMAN, LUNDBERG & WOESSNER, P.A., P.O. BOX 2938, MINNEAPOLIS, MN, 55402, US

(73) Assignee: Regents of the University of Minnesota, St. Paul, MN (US)

(21) Appl. No.: 12/837,224

(22) Filed: Jul. 15, 2010

Related U.S. Application Data
(60) Provisional application No. 61/225,594, filed on Jul. 15, 2009.

(51) Int. Cl. A61M 29/02

(52) U.S. Cl. 604/514; 606/199; 604/104

(57) ABSTRACT

Devices, methods and kits which, when installed or used, urge or maintain patency of sinus ostia, such as the ostium between a nasal passageway and a maxillary sinus, while maintaining some function of cilia extending within or near the ostium. In various examples, the devices comprise a first portion, a second medial portion including a partially open cross-sectional configuration, and a third portion including one or more flexible and outwardly-biased projections. The partially open cross-sectional configuration can maintain a track of uncovered cilia within and near the ostium. In some examples, the devices can include an asymmetric body configuration. In some examples, the first portion includes a U-shaped configuration and the third portion includes at least two flexible and outwardly-biased projections. In some examples, the first, second or third portion includes a medication for delivery into sinus tissues.

### CLAIM OF PRIORITY

This non-provisional patent application claims the benefit of priority under 35 U.S.C. § 119(e) to U.S. Provisional Patent Application Ser. No. 61/225,594, entitled “IMPLANTABLE DEVICE FOR MAXILLARY SINUS OSTIUM,” (Attorney Docket No. 600.793PRV), filed on Jul. 15, 2009, which is hereby incorporated by reference in its entirety.

### TECHNICAL FIELD

This patent document pertains generally to devices, methods and kits to treat sinusitis. More particularly, but not by way of limitation, this patent document pertains to devices, methods and kits which, when installed or used, urge or maintain patency of sinus ostia.

### BACKGROUND

“Sinusitis” refers to inflammation of one or more nasal sinus cavities, which are moist, hollow spaces in the bones of the skull. Sinusitis affects millions of people each year. According to the National Ambulatory Medical Care Survey (NAMCS), approximately 14% of adults report having at least one yearly episode of sinusitis. Further, sinusitis is believed to be one of the top five most common diagnoses for which antibiotics are prescribed.

There are four pairs of sinuses: frontal, maxillary, ethmoid, and sphenoid. The sinuses are located behind the eyebrows, cheekbones, and nose. The sinuses help moisten and warm air that is filtered by the nose, serving to protect the lungs. The sinus cavities, nose, and lungs are lined with mucous membranes, which protect the airways by trapping irritants that are inhaled. Tiny hair-like filaments called cilia are in constant movement and sweep mucus and the trapped irritants out of the airways and nasal passages. In each of the sinuses, the mucus drains out of a tiny opening called the ostium.

The ostium of any sinus cavity can get plugged. When this blockage prevents the flow of mucus, the pressure builds up, leading to sinusitis typified by inflammation and pain. If the blocked sinus becomes infected with bacteria, the sinus becomes even more inflamed and painful. Symptoms of sinusitis can include, among other things, facial pain or pressure, discolored mucus, a diminished sense of smell, cough, headache, bad breath, fever, toothache, pressure in the ears and fatigue.

### Overview

The present inventors have recognized, among other things, that many subjects suffering from sinusitis can benefit from devices that are configured to urge or maintain patency of sinus ostia without circumferentially compromising ciliary function, thereby promoting natural healing. The present inventors have further recognized a need for sinus specific tools that can effectuate clinical-based therapy, and comfortably accommodate the natural nasal and sinus geometries in maneuvering and placement of sinus ostium patency devices. Further yet, the present inventors have recognized that medicated sinus ostium patency devices can offer site specific drug treatment, if deemed necessary or beneficial.

The present devices are configured to open and maintain sinus ostia in a manner that allows sinus cavities to drain without compromising the physiological function of cilia. Because each maxillary sinus cavity, for example, has its ostium at the cranial end of the cavity, the cilia work against gravity much of the time in moving mucus from within the maxillary sinus toward the ostium. The design of the present devices include an open or partially open cross-sectional configuration to expose cilia near and within the ostium, thus allowing for normal or near normal ciliary function.

To better illustrate the devices, methods and kits disclosed herein, a non-limiting list of examples is provided here:

In Example 1, a device configured for placement in a sinus ostium comprises a first portion; a second medial portion including a partially open cross-sectional configuration, the partially open cross-sectional configuration maintaining a track of uncovered cilia within the sinus ostium; and a third portion including one or more flexible and outwardly-biased projections, each of the projections having a first end integrally attached to the second medial portion, wherein the first portion, the second medial portion and the third portion urge or maintain a patency of the sinus ostium.

In Example 2, the device of Example 1 optionally comprises one or more corrugations or lumens along a lateral surface of the first portion, the second medial portion or the third portion, the one or more corrugations or lumens facilitating drainage flow through the sinus ostium.

In Example 3, the device of any one or any combination of Examples 1 and 2 is optionally configured such that a width of the first portion and a width of the third portion are greater than a width of the second medial portion.

In Example 4, the device of any one or any combination of Examples 1-3 is optionally configured such that the first portion includes a U-shaped configuration and the third portion includes at least two flexible and outwardly-biased projections.

In Example 5, the device of Example 4 is optionally configured such that the U-shaped configuration includes an outward curvature configured to seat against a middle sinus concha and within a nasal passageway.

In Example 6, the device of any one or any combination of Examples 1-5 is optionally configured such that the first portion includes a conchal spacing structure.

In Example 7, the device of any one or any combination of Examples 1-6 is optionally configured such that the third portion includes an expandable interconnecting structure between a first and a second flexible and outwardly-biased projection.

In Example 8, the device of any one or any combination of Examples 1-7 is optionally configured such that one or more of the first portion, the second portion or the third portion includes a polymeric material.

In Example 9, the device of any one or any combination of Examples 1-8 is optionally configured such that one or more of the first portion, the second portion or the third portion includes a metallic material.

In Example 10, the device of any one or any combination of Examples 1-9 is optionally configured such that one or more of the first portion, the second portion or the third portion includes a biodegradable material.

In Example 11, the device of any one or any combination of Examples 1-10 is optionally configured such that one or more of the first portion, the second portion or the third portion includes a medication for delivery into the sinus ostium or an associated sinus cavity.

In Example 12, a method comprises placing a sinus ostium patency device extending from a first portion to a third portion within or near a sinus ostium, including placing one or more flexible projections of the third portion at least partially in a maxillary sinus, extending a second portion, intermediate the first and third portions, through the sinus ostium, and placing the first portion under or near a middle sinus concha; and urging or maintaining an unobstructed channel or passageway through the sinus ostium without circumferentially compromising ciliary function.

In Example 13, the method of Example 12 optionally comprises releasing one or more pharmaceuticals or medications into the maxillary sinus or sinus ostium using the sinus ostium patency device.

In Example 14, the method of any one or any combination of Examples 12 and 13 is optionally configured such that placing the sinus ostium patency device includes orienting an asymmetric device body relative to a subject's ostium geometry.

In Example 15, the method of any one or any combination of Examples 12-14 is optionally configured such that placing the sinus ostium patency device includes moving two flexible projections from a collapsed position to an outwardly-biased position, partially flared against a wall of the sinus ostium.

In Example 16, the method of any one or any combination of Examples 12-15 optionally comprises reducing an ostium obstruction associated with a concha located adjacent the ostium, including using an extension structure of the sinus ostium patency device to space the concha from the ostium.

In Example 17, the method of any one or any combination of Examples 12-16 is optionally configured such that urging or maintaining the unobstructed channel or passageway includes allowing the sinus ostium patency device to self-deploy.

In Example 18, the method of any one or any combination of Examples 12-17 is optionally configured such that urging or maintaining the unobstructed channel or passageway includes using an expandable structure to outwardly-bias the sinus ostium patency device.

In Example 19, a kit comprises a device of any one or any combination of Examples 1-11; and a set of instructions for using the device to urge or maintain the patency of an ostium associated with a maxillary sinus, a frontal sinus or an ethmoid sinus, without circumferentially compromising ciliary function within the ostium.

In Example 20, the kit of Example 19 optionally further comprises a treatment and placement device including a catheter having a side portal and a probe, the probe doubled-backed within the catheter and configured to partially exit via the side portal.

In Example 21, the device, method or kit of any one or any combination of Examples 1-20 is optionally configured such that all elements or options recited are available to use or select from.

These and other examples, advantages, and features of the present devices, methods and kits will be set forth in part in following Detailed Description. This Overview is intended to provide non-limiting examples of the present subject matter—it is not intended to provide an exclusive or exhaustive explanation. The Detailed Description is included to provide further information about the present devices, methods and kits.

### BRIEF DESCRIPTION OF THE DRAWINGS

In the drawings, like numerals can be used to describe similar components throughout the several views Like numerals having different letter suffixes can be used to represent different instances of similar components. The drawings illustrate generally, by way of example, but not by way of limitation, various embodiments discussed in the present document.

FIG. 1 illustrates an example schematic view of anatomical tissue structures including the maxillary, frontal, and ethmoid sinus cavities, such tissue structures providing a suitable environment in which a sinus ostium patency device, as constructed in accordance with at least one embodiment, can be used.

FIGS. 2A-3B illustrate isometric and transverse cross-sectional views of two sinus ostium patency device embodiments, each embodiment including an at least partially open cross-sectional configuration to promote cilia function.

FIG. 4 illustrates an implanted view, from a transverse plane, of another sinus ostium patency device, as constructed in accordance with at least one embodiment.

FIG. 5 illustrates an isometric view of another sinus ostium patency device, as constructed in accordance with at least one embodiment.

FIG. 6 illustrates an isometric view of another sinus ostium patency device, as constructed in accordance with at least one embodiment.

FIG. 7 illustrates an isometric view of another sinus ostium patency device, as constructed in accordance with at least one embodiment.

FIGS. 8A-8B illustrate end and side views of another sinus ostium patency device, as constructed in accordance with at least one embodiment.

FIGS. 9A-25B illustrate isometric and transverse cross-sectional views of various sinus ostium patency device embodiments, each embodiment including an at least partially open cross-sectional configuration to promote cilia function.

FIGS. 26A-26G illustrate cross-sectional views of various sinus ostium patency device embodiments, each embodiment including an at least partially open cross-sectional configuration to promote cilia function.

FIGS. 27A-27G illustrate cross-sectional views of various sinus ostium patency device embodiments, each embodiment including an at least partially open cross-sectional configuration to promote cilia function.

FIGS. 28A-28B illustrate schematic views of a distal end of an example placement device and a patency device positioned near and within a sinus ostium, the placement device and the patency device as constructed in accordance with at least one embodiment.

FIGS. 29A-29C illustrate schematic views of an example treatment and placement device and a patency device positioned near and within a sinus ostium, the treatment and placement device and the patency device as constructed in accordance with at least one embodiment.

### DETAILED DESCRIPTION

The present invention provides implantable devices, methods and kits for insertion or placement within a sinus ostium for maintaining an unobstructed channel there-through. The present inventors have discovered that implantable devices can be constructed for placement within a maxillary sinus ostium of a subject so-as-to urge or maintain an unobstructed channel or passageway without circumferentially compromising ciliary function, thereby promoting natural healing. The implantable devices can accomplish either the urging or maintenance, or both, of the ostium channel and the reduction of physical obstruction associated with a concha located adjacent the ostium. The devices can be constructed from permanent (i.e., non-biodegradable), partially biodegradable or fully biodegradable materials, and can be utilized for immediate or extended release of pharmaceutical or other medication delivery into a sinus. In some examples, the sinus ostium patency devices can be asymmetrically constructed for a particular placement orientation relative to a subject's natural anatomical geometry.

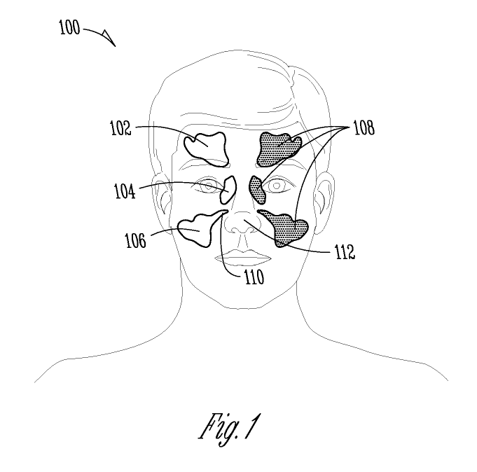

To facilitate understanding of the physiology associated with the present devices, methods and kits, FIG. 1 illustrates anatomical tissue structures 100 associated with sinusitis. There are four different pairs of sinuses—the frontal sinuses 102, the ethmoid sinuses 104, the maxillary sinuses 106, and the sphenoid sinuses (located more toward the back of the head than the other sinuses). Normally, sinuses are filled with air, but when sinuses become blocked and filled with fluid, pathogens can grow and cause an infection. In FIG. 1, the sinuses on the (reader's) right side are shown as inflamed, infected and experiencing sinusitis 108.

Sinusitis 108 can be acute or chronic. Acute sinusitis usually lasts for approximately 3 weeks, but can persist for as long as 3 months. Acute sinusitis is usually caused by a viral respiratory infection. Subjects with acute or recurrent acute sinusitis are typically treated with antibiotics or over-the-counter decongestants.

Chronic sinusitis lasts longer than three months and does not respond well to conventional antibiotic treatment. Chronic sinusitis can also be caused by infection, but is more often caused by inflammation and blockage due to allergies or a physical obstruction (e.g., collapsed bone or cartilage structures or foreign objects). Traditionally, chronic sinusitis is treated using Functional Endoscopic Sinus Surgery, commonly known as a FESS procedure. During a traditional FESS procedure, a physician caregiver resects or removes bone and tissue to enlarge a sinus ostium of interest to restore adequate cavity drainage. Removal of bone and tissue can lead to considerable post-operative pain.

A technological concept of the present devices, methods and kits is to open and maintain a blocked maxillary sinus ostium 110 extending between a cavity of the maxillary sinus 106 and a nasal passage 112, for example, to reduce mucus pressure build-up and the resulting pain experience by a subject without removing bone or tissue. Non-removal procedures, by nature, are associated with less healing times and discomfort than invasive removal procedures, and thus, provide advantages over such procedures.

The present implantable devices have been designed such that, when positioned or implanted, they can urge or maintain the patency of the maxillary sinus ostium 110 allowing for drainage without circumferentially compromising ciliary function. FIGS. 2A-27G illustrate various implantable device embodiments configured to address the pathophysiology of sinusitis 108 by opening and maintaining the patency of a desired ostium 110 in a manner that allows the sinuses to drain without compromising the physiological function of cilia 222 located at or near the treated ostium 110. The connection from the cavity of a maxillary sinus 106 to the nasal cavity 112 includes a short, upward sloping channel 220. In normal maxillary sinus drainage, mucus is swept up this channel 220, against gravity, and out the ostium 110 by the cilia 222.

When implanted and allowed to expand or deploy into position, the present devices are intended to open and maintain patency of the sinus ostium 110, facilitating mucosal drainage and healing. To accomplish this, as shown in the examples of FIGS. 2B, 3B, 4, 9B, 10B, 11B, 12B, 13B, 14B, 15B, 16B, 17B, 18B, 19B, 20B, 21B, 22B, 23B, 24B and 25B, a third portion 204 of a device body 202 can sit at least partially in the maxillary sinus 106, while a second portion 206 of the device body 202 extends through the maxillary sinus ostium 110 and a first portion 208 of the device body 202 sits under or near a portion of a middle sinus concha 224. Outside the ostium 110, there can be an addition or extension to the device body 202 to maintain the middle concha 224 at a spaced-apart distance from the ostium 110 opening. Examples of these additions can include a hood, a horizontal U-shape, or a downward angled extension. The first 208 and third 204 portions of the device body 202 can be flared or biased-outward to hold the device in place.

As illustrated in the examples of FIGS. 2A-27G, the body 202 of each sinus ostium patency device can include a cross-sectional shape that opens and maintains the channel 220 from the maxillary sinus 106 into the nasal passageway 112, while maintaining a track or region of uncovered sinus mucosa between the two cavities. The mucosa track or region allows the cilia 222 therein to continue to provide movement of mucus and fluids out of the maxillary sinus 106. Optionally, fenestrations, corrugations, or lumens can be present along the length of the device body 202 to facilitate mucus and fluid drainage.

In the examples of FIGS. 2A-2B and 3A-3B, the sinus ostium patency device 200, 300 extends from a U-shaped first portion 208 to a third portion 204 including two flexible projections 212, 312 movable between collapsed and outwardly-biased positions. The third portion 204 and its two flexible projections 212, 312 can sit at least partially in the maxillary sinus 106, while a second portion 206 of the device body 202 extends through the maxillary sinus ostium 110 to the U-shaped first portion 208 disposed under or near a middle sinus concha 224 within a nasal passageway 112. The device body 202 can have a cross-sectional configuration that opens the sinus ostium 110 from the maxillary sinus 106 into the nasal passageway 112, while maintaining a track or region 250, 350 of uncovered sinus mucosa and cilia 222 between the two cavities.

Prior to insertion into the maxillary sinus ostium 110, the patency device 200, 300 can be compressed or folded upon itself within a placement device. When deployed, the device 200, 300 can expand and hold open the sinus ostium 110, facilitating mucosal drainage and healing. To hold the device 200, 300 in place and inhibit migration, the two flexible projections 212, 312 of the third portion 204 can be flared outward against a wall of the maxillary sinus 106, and the U-shaped first portion 208 can include an outward curvature 214, 314 configured to seat against the middle sinus concha 224 and nasal side of the ostium 110, and within the nasal passageway 112.

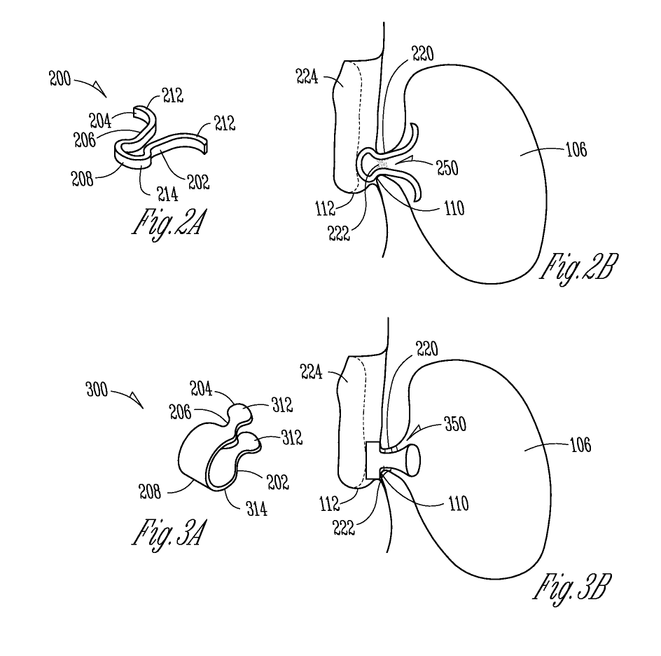

In the examples of FIGS. 4, 5 and 6, the sinus ostium patency device 400, 500 and 600 extends from a first portion 208 including a flange 416, 516 and 616 to a third portion 204 including a portion of at least two flexible projections 412, 512 and 612 movable between collapsed and outwardly-biased positions. The number and configuration of the flexible projections 412, 512 and 612 can vary. Each of the flexible projections 412, 512 and 612 can have a first end 430, 530 and 630 integrally attached to a second medial body portion 206 and a second end 432, 532 and 632. The flexible projections 412, 512 and 612 can collectively form at least a portion of the second medial portion 206 and the third portion 204 of the device 400, 500 and 600.

The flange 416, 516 and 616 or the first portion 208 can have a variety of forms, provided the structure facilitates placement of the device body 202 within an ostium 110 and reduces the likelihood of migration of the device within the ostium channel 220. Although depicted as a contiguous circumscribing rim, the term “flange” is intended to include other structures, such as one or more protrusions or extensions from the first portion 208, both contiguous and broken (or partial).

In the example of FIG. 5, the first portion 208 of the device body 202 includes a conchal spacing structure 518. The conchal spacing structure 518 can be constructed as part of the flange 516 or, alternatively or in addition, can be constructed as a separately protruding structure from the first portion 208 of the device body 202. Advantages afforded by a flange 416, 516 and 616, conchal spacing structure 518, or both, include facilitating placement of the device body 202 with an ostium 110 and reducing the likelihood of migration of the device body 202 within a ostium channel 220 toward the sinus side, as well as, reducing or preventing conchal occlusion or obstruction of the ostium 110 on the nasal side. In some examples, the device body 202 can include one or more surface characteristics (e.g., teeth) or suture securing means (e.g., eyelets) to engage adjacent tissue structures, thereby further securing an implanted device placement.

As shown in the example of FIG. 6, a width W1 of the first portion 208 of the device body 202 can be greater than a width W2 of the second portion 206. In some examples, W2 can be slightly greater than that of an adjacent ostium channel 220, such that the device body 202 can be partially held in place by the channel walls. A width W3 of the third portion 204 of the device body 202 can be greater than that of W2, and optionally, W1. In various examples, the device body 202 can comprise an overall width ranging from between about 1 mm to about 15 mm, and an overall length ranging from between about 2 mm to about 20 mm. In some examples, the device body 202 can be structured to have a fixed cross-sectional dimension (e.g., width or diameter). In some examples, the device body 202 can be structured to have a variable width or diameter in addition to the widths defined as W1, W2 and W3, thereby allowing the device to accommodate natural anatomical variability in subjects.

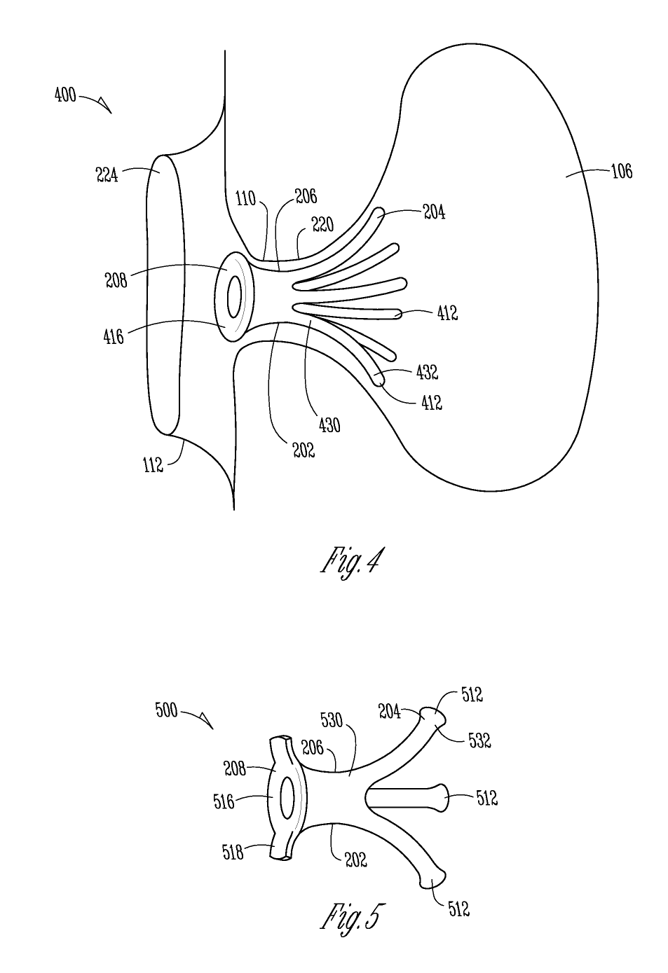

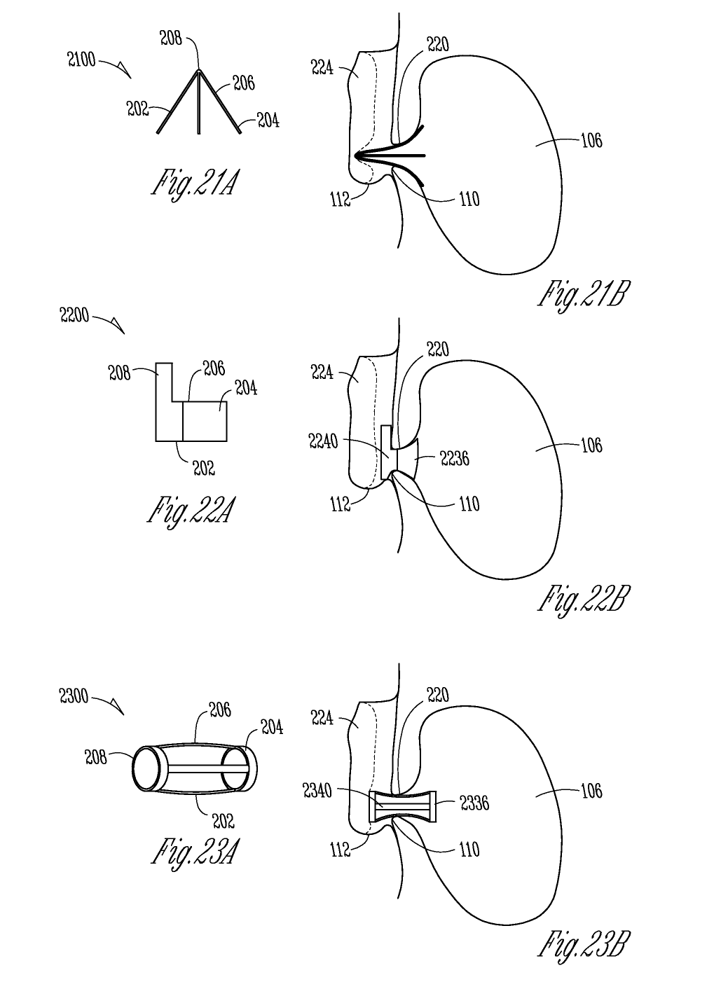

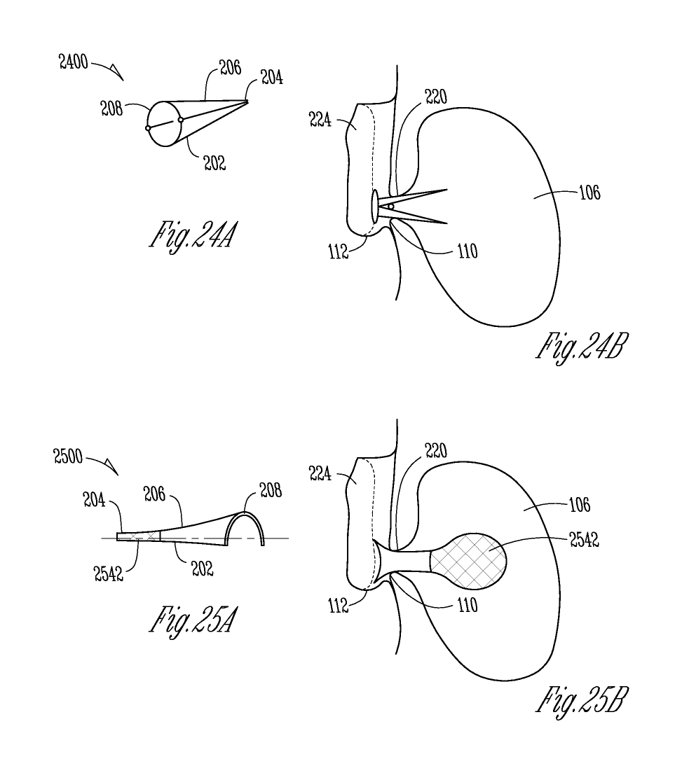

Although illustrated as having a continuous, tubular configuration with a circular cross-sectional shape in the FIGS. 4, 5 and 6, the cross-sectional shape of the device body 202 can vary and can be broken, as shown in the examples of FIGS. 7 and 26A-26G, and non-circular, as shown in the examples of 26B-26E, 26G, 27B-27E and 27G. An advantage of a non-continuous, broken device body 202 design can include increased conformability to the natural anatomical geometry of a subject. In addition, broken device bodies 202 can provide enhanced adaptability of fit for a single manufacturing design across a multitude of subjects. Further, the natural ciliary action within the ostium channel 220 can occur unobstructed along the open track area or region, such as the open “C” 732 of device 700 shown in FIG. 7, thereby facilitating transport of mucous out from the maxillary sinus cavity 106 through the ostium 110.

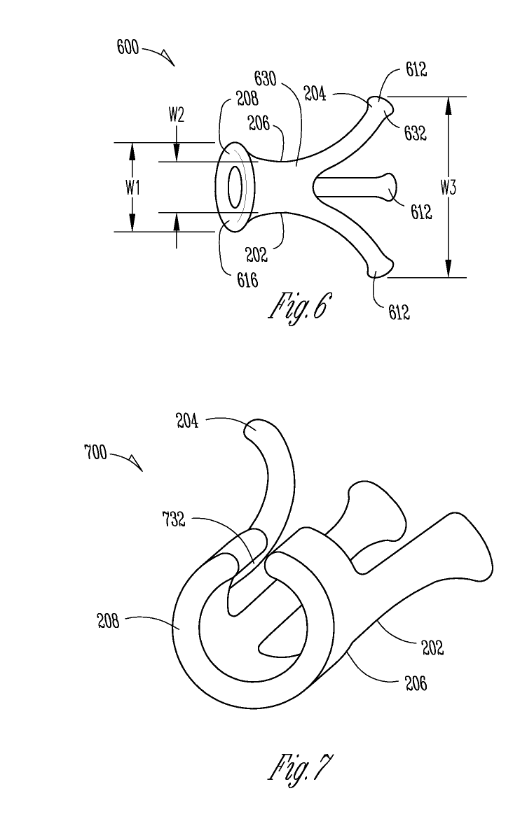

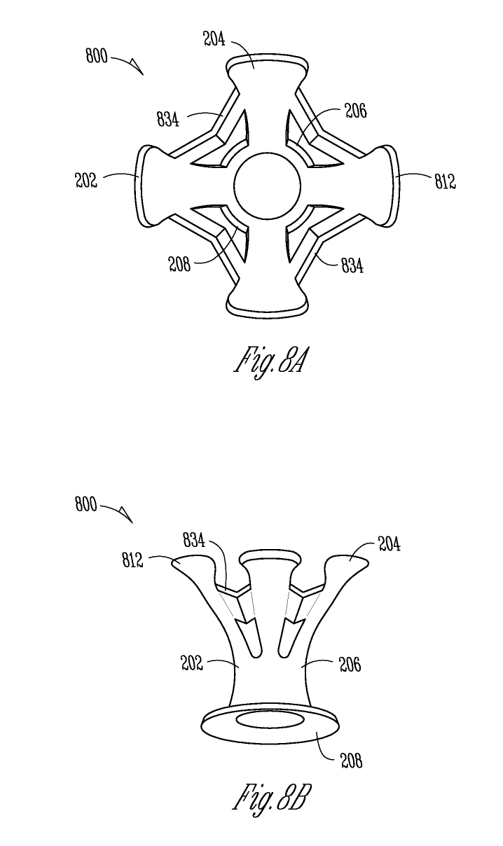

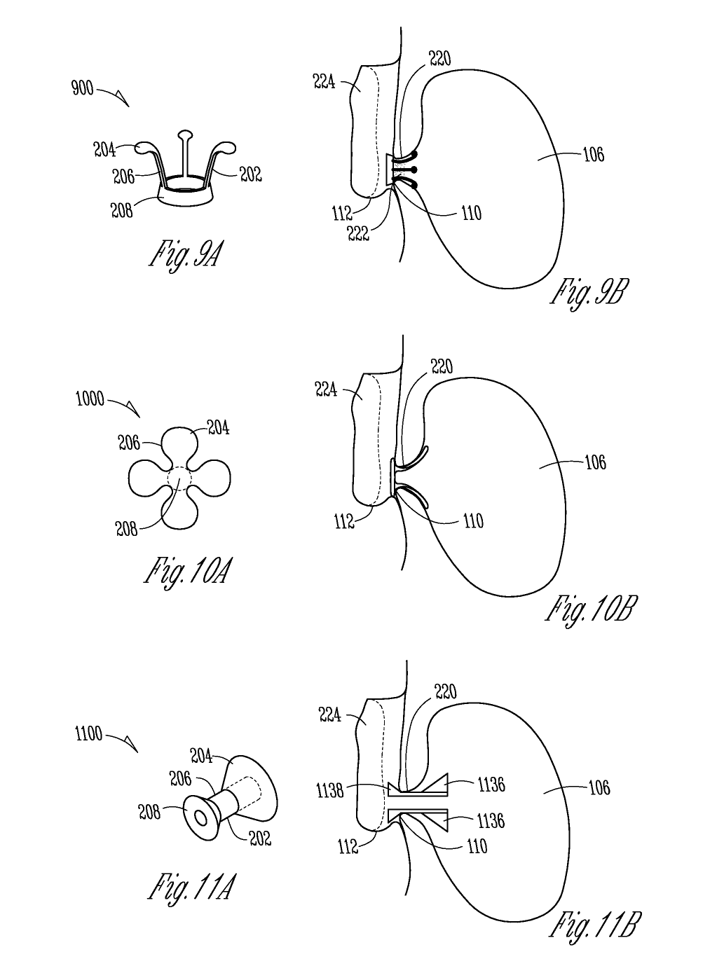

The third portion 204 of the device 800 can optionally include one or more expandable interconnecting structures 834, as shown in FIGS. 8A and 8B. The interconnecting structures 834 can connect two or more of the flexible projections 812 of the device body 202. In addition to enhancing the overall structural integrity of the device body 202, the expandable interconnecting structures 834 can further enhance and control the outward expansion and spatial interrelationship between the flexible projections 812 both during and after placement into an ostium 110. In some examples, the expandable interconnecting structures 834 can be in the form of a circumscribing band connecting all or some of the flexible projections 812 or “accordion” (in either the radial or axial directions) to permit minimally invasive instrumentation (e.g., a reduced diameter catheter) for delivery and placement of a device within an ostium channel 220.

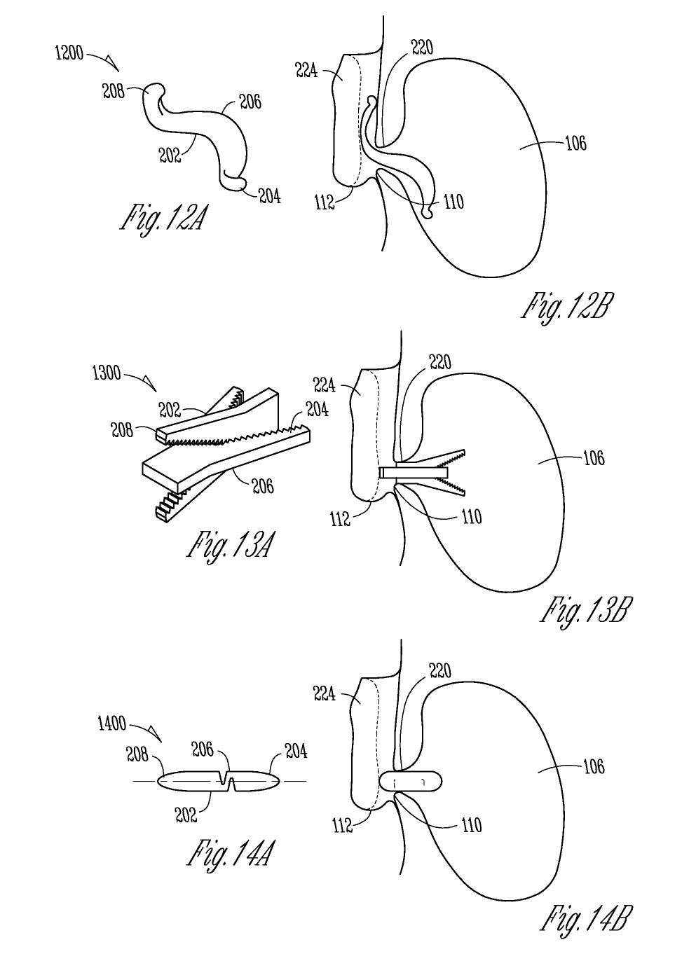

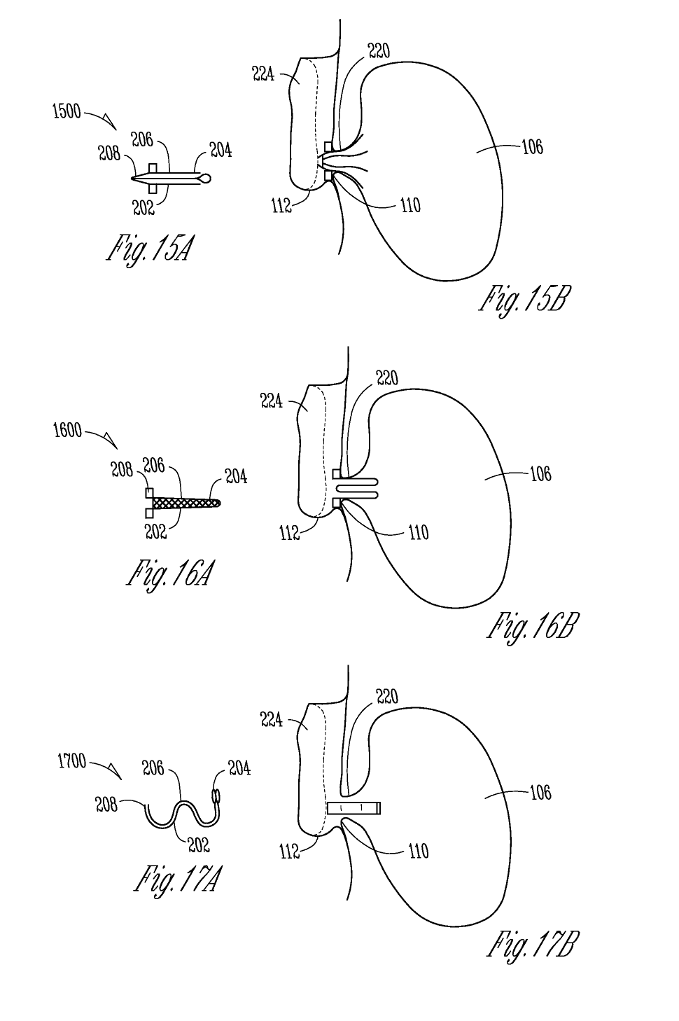

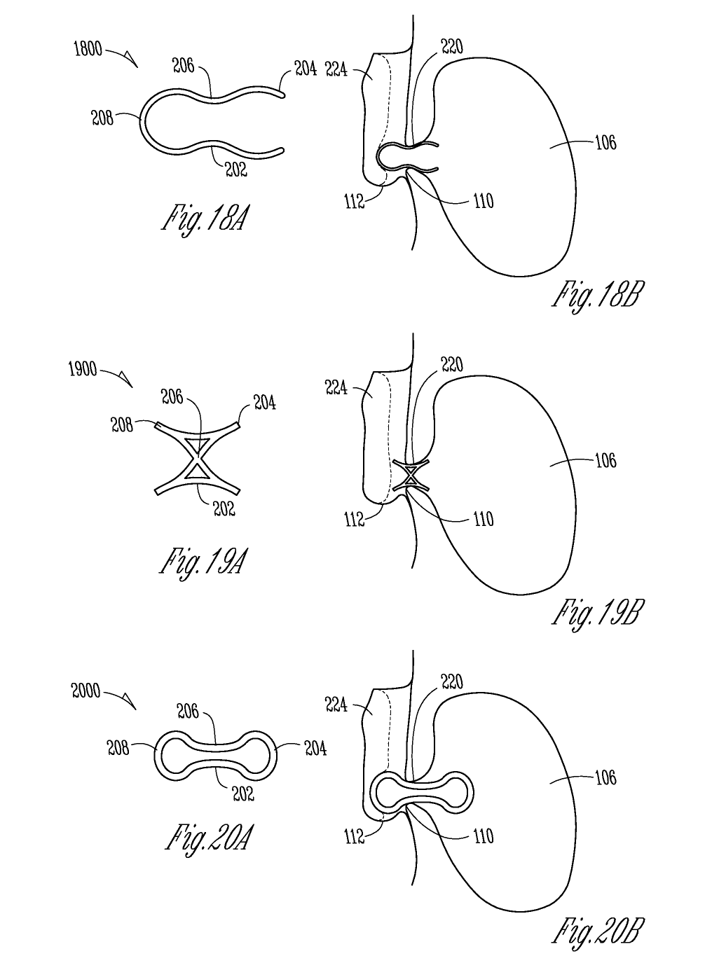

Other options and illustrations for the sinus ostium patency devices include the following. In the example of FIGS. 9A and 9B, the device 900 includes a crown-like configuration. In the examples of FIGS. 10A and 10B, the device 1000 includes wire petals. In the example of FIGS. 11A and 11B, the device 1100 includes a biodegradable flare or flange that can degrade over time, thereby allowing a non-biodegradable portion 1138 of the device to fall out of the ostium 110 via the nasal passageway 112. In the example of FIGS. 12A and 12B, the device 1200 includes an S-shaped clip. In the example of FIGS. 13A and 13B, the device 1300 includes one or more ratcheting wedges. In the example of FIGS. 14A and 14B, the device 1400 includes a retaining mechanism configuration, which can be released via a clip or center wire, for example. In the example of FIGS. 15A and 15B, the device 1500 includes a push-through pin deployment mechanism. In the example of FIGS. 16A and 16B, the device 1600 includes a deployable mesh. In the example of FIGS. 17A and 17B, the device 1700 includes a deployable spring. In the example of FIGS. 18A and 18B, the device 1800 includes a shape-memory (e.g., Nitinol) clip. In the example of FIGS. 19A and 19B, the device 1900 includes a shape-memory car-jack design. In the example of FIGS. 20A and 20B, the device 2000 includes a shape-memory closed wire. In the example of FIGS. 21A and 21B, the device 2100 includes a shape-memory umbrella design. In the example of FIGS. 22A, 22B, 23A and 23B, the device 2200, 2300 includes a shape-memory material 2240, 2340 in combination with a biodegradable material 2236, 2336. In the example of FIGS. 24A and 24B, the device 2400 includes a hinged, jaw-like design. In the example of FIGS. 25A and 25B, the device 2500 includes a deployable balloon

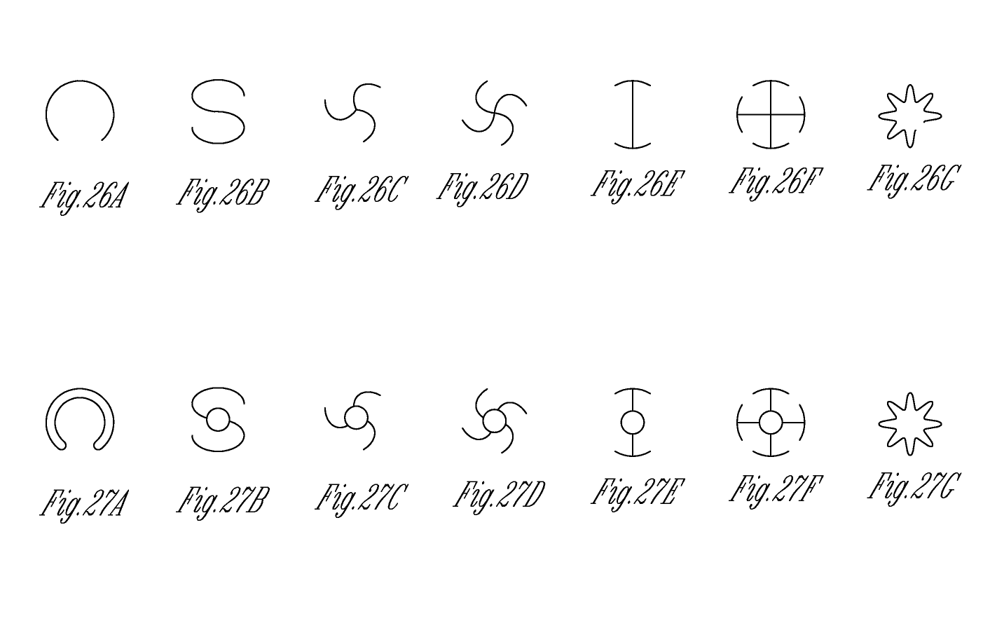

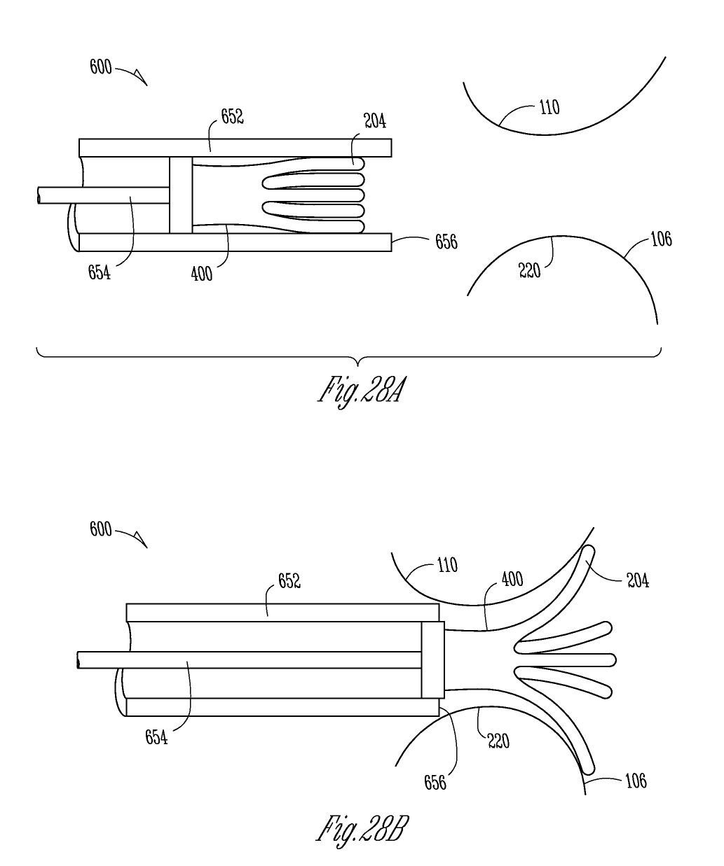

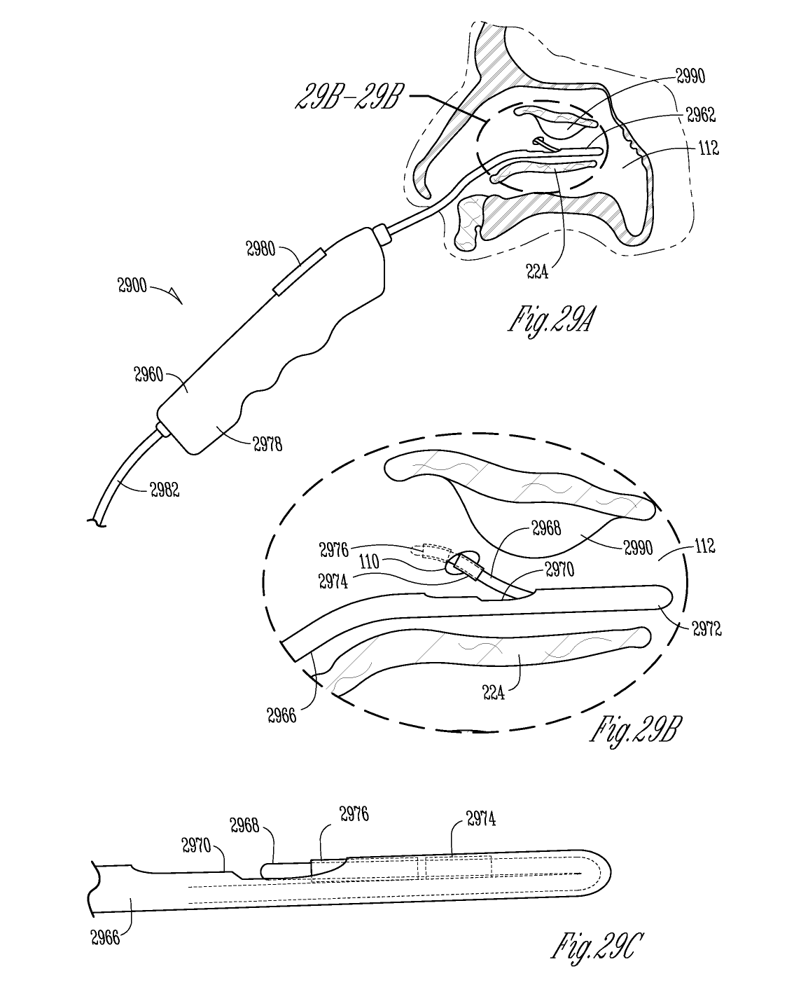

In general, the sinus ostium patency devices can have any overall configuration that is insertable and securable with a sinus ostium 110, while maintaining some function of cilia 222 extending within or near the ostium. Example cross-sectional configurations for the devices are shown in FIGS. 26A-26G (without defined lumens) and FIGS. 27A-27G (with defined lumens). In various examples, the overall configuration and dimensions of the patency devices are circular or partly circular to be consistent with the natural ostium channel 220 configuration.

The sinus ostium patency devices can be made from a variety of materials. In some examples, the devices can be constructed from a single uniform material. In other examples, the device can be constructed from two or more different materials having one or more differing chemical or physical properties. For example, the devices can be constructed from a polymeric material embedded with a radiopaque material, or a metallic lattice coated with a polymer or plastic material. Any part of the devices can include a radiopaque marker as either a solid marker attached to a portion of the devices or as fine powder mixed with a device body material during construction. Whether constructed from a single material or multiple materials, the material(s) employed in the devices should exhibit sufficient structural integrity to encourage and maintain diametrical expansion of ostium 110 tissue, while reducing likelihood of collapse. Optionally, the devices can be composed of variety of rigid or semi rigid materials, more preferably, however, flexible and semi-rigid materials can be used to enhance comfort and adaptability to anatomical variation of a subject's ostium 110.

The sinus ostium patency devices can be constructed as a long-term resident implant, or alternatively as a temporary biodegradable implant. To this end, the devices can be constructed from permanent non-biodegradable materials, partially biodegradable materials, or fully biodegradable materials. In some examples, the device is made from a biodegradable material and is intended for temporary use. Biodegradable materials can allow a subject to make a single office visit to his/her caregiver to insert the device. Over time, the biodegradable material can degrade and extraction may not be needed, thus avoiding subsequent office visits. Suitable materials include, but are not limited to, metals, metallic alloys, plastics and polymeric materials. Examples of metals and metallic alloys that can be used include shape memory alloys (SMAs), such as Nitinol, and biodegradable metals, such as magnesium or iron. Plastics and polymeric materials that can be used include, but are not limited to, PLLA, PGA, DLLA, PLGA, PDGLA, PLDLA, PLC, polyethylene (PE), polyglycolic acid, polylactic acid (PLA), PEEK or silicone. In some examples, a foam material or a material expandable from a first dry state to a second, wet state can be used.

The devices can comprise materials and compositions that can affect immediate or extended release of pharmaceuticals or other medications into a sinus cavity, to sinus tissues or to an uncinate process. In some examples, the devices can be structured with a permanent portion or lattice coated with a biodegradable material, which incorporates an active pharmaceutical agent and an immediate or prolonged release excipient composition. Pharmaceutical agent(s) can be applied to the biodegradable implant on its surface or via impregnation to provide an agent therapy elution medium. Among other things, the pharmaceutical or other agent can include steroids or NSAIDs to decrease swelling, adrenergics to decrease blood flow and decrease swelling, antihistamines or antilekotrienes to block the affects of these chemicals, mucus thinners, or antibiotics or an antiseptic to fight infection. Over time, these pharmaceuticals or other agents can dissolve and flow into a sinus cavity providing extended pharmaceutical therapy.

As discussed above, the sinus ostium patency devices, once placed in situ and deployed in a maxillary sinus 106 ostium 110, for example, can provide an unobstructed channel 220 through the ostium 110. When deployed, the device can expand and hold open the sinus ostium 110, facilitating mucosal drainage and healing. In some examples, the device is self-deploying. In some examples, the device can be deployed using an expandable structure (e.g., an inflatable member, such as a balloon). In other examples, the device is sheath deployed, liquid expandable, inflatable, opened with the removal of a hinge pin, expands as a bonding material dissolves, is UV cured in place, or is wedge expandable. The patency devices of the invention can be implanted using conventional surgical techniques, equipment and instrumentation. In addition, a plunger-like placement device 600, such as is shown in FIGS. 6A and 6B, can be used to place a patency device of the invention into the ostium 110. Prior to insertion into a sinus ostium, the patency device can be compressed or folded upon itself within a placement device.

The placement device 600 can include a catheter 652 and plunger 654 assembly to retain and expel a sinus ostium patency device, such as device 400 shown in FIG. 4, out from a distal tip 656 of the placement device. The distal tip 656 of the placement device can be positioned into the ostium 110 of the subject, and the patency device 400 can be expelled by distal movement of the plunger 654, thereby expelling the patency device 400 into the ostium and permitting expansion of the device's 400 third portion 204 (shown having a plurality of expandable legs).

Removable patency device embodiments can include a removal structure, which can be accessed using approaches from the nasal side toward the ostium 110. Suitable removal structures include, but are not limited to, tethers, tabs, loops, hooks, cords, and the like. A cord, for example, can be attached between left and right implanted device so that a portion passes through a puncture in the nasal septum or out through the nostrils. A variety of removal structures can be employed provided a surgical grasping instrument can access and grasp the removal structure to withdrawn the patency device from the ostium 110 and sinus of the subject. When present on the patency device, the conchal spacing structure 518 (FIG. 5) can be used or structured to facilitate removal of the device.

In a preferred example, the patency devices can be inserted and deposited in the ostium 110 of a maxillary sinus 106 (FIG. 1) using a treatment and placement device 2900, such as that shown in FIGS. 29A-29C and described in commonly-owned Arcand, et al., U.S. patent application Ser. No. ______, entitled “TREATMENT AND PLACEMENT DEVICE FOR SINUSITIS APPLICATIONS,” filed concurrently herewith, the entire text of which is incorporated herein by reference. The treatment and placement device 2900 has been designed to navigate the bony and cartilaginous obstructions within the nasal passageway 112 and access a maxillary sinus ostium 110 for delivering a therapy, ostium dilation, or positioning of a patency device in a retrograde manner.

The treatment and placement device 2900 can extend from a first portion 2960 to a second portion 2962, and can include a catheter 2966 that houses a probe 2968. The probe 2968 can be doubled-backed within the catheter 2966 so that its tip faces towards the first portion 2960 of the device 2900. This is enabled through the use of a side or lateral portal 2970, in lieu of a distal tip 2972 portal, which can be configured for delivery of a therapy, a dilator (e.g., balloon), or a patency device 2974. The overall catheter 2966 can be shaped to facilitate positioning alongside the sinus ostium 110 by providing an angle configured to enter the nostril and slide along the middle sinus concha 224. The probe 2968 that exits the catheter 2966 via the lateral portal 2970 can be shaped so that it bends toward the sinus ostium 110 and uses the uncinate process 2990 to aid in advancement into the ostium 110. Location monitoring of the catheter 2966 and probe 2968 during a procedure can, in some examples, be determined using tactile feedback.

The probe 2968 can be separately actuated for retrograde movement without requiring movement of the catheter 2966. A balloon or other dilator 2976 can be advanced via actuation of the probe 2968 and positioned against a blocked sinus ostium 110. When the balloon or other dilator 2968 is inflated (e.g., via a lumen within the probe 2968), the fragile bones of the sinus ostium 110 can be permanently or sufficiently moved aside to open up air and mucus flow. Optionally, the device 2974 can then be advanced via actuation of the probe 2968 and inserted into a newly-unblocked sinus ostium 110. Additionally, a lumen within the probe 2968 can be used to deliver therapeutic pharmaceutical agents, irrigation fluid, or other therapies directly into the maxillary sinus 106. The lumen can also house an actuation wire or electrical wires that can be used to power a therapy or release an implant. The first portion 2960 of the treatment and placement device 2900 can include a handle 2978 with a slide or other mechanism 2980 to actuate the probe 2968 out of, and back into, the catheter 2966. Additional controls can exist to release, actuate or power a therapy, dilator, and/or implant. Fluidic port(s) 2982 in the handle 2978 can provide attachment to pressure or fluid delivery applications.

The treatment and placement device can, in some examples, include a second portion 2962 having a generally flattened configuration. The catheter 2966 and probe 2968 can be made from a bendable or flexible material so-as-to be atraumatic to bodily tissue. Any part of the treatment and placement device 2900 can include a radiopaque marker as either a solid marker attached to a portion of the device or as fine powder mixed with the catheter or probe material during construction. Additional features for the treatment and placement device 2900 include: pre-loading of a diagnostic, therapeutic or other active agent; a reservoir (e.g., to store active agent(s)); inclusion of an endoscope of other internal viewing device (e.g., a light-emitting means); coating with a hydrophilic material; configuration for placement within a frontal sinus; a hose for draining and/or rinsing a maxillary sinus; or specimen capturing jaws for removal of a tissue sample (biopsy).

A retail kit may also be packaged for consumer purchase. The kit can include one or both of a sinus ostium patency device and a treatment and placement device. The kit can also include a set of instructions for using the patency device or the treatment and placement device. In some examples, the kit includes one or more separate tools for withdrawing the sinus ostium patency device.

Closing Notes:

Devices, methods and kits which, when installed or used, urge or maintain patency of sinus ostia without circumferentially comprising ciliary function are disclosed. Advantageously, the present devices, methods and kits promote healing by allowing cilia to function normally, or near normally, in the sinus ostia. The devices of the present invention afford several ways to accomplish or accommodate this, including but not limited to, selection of spacing between flexible projections, reducing the length of a second portion of the device, employing a narrow projection configuration, and varying the number of projects. In various examples, the device is configured for placement within a maxillary sinus ostium. In some examples, medication devices can be used to offer site specific drug treatment, if necessary or beneficial.

Advantageously, the present devices, methods and kits are believed to lower the overall cost of sinus care and prevent long term health issues by (a) reducing the dependence on systemic pharmaceuticals and other medications, (b) avoiding invasive, cutting-based procedures for device placement with an ostium, and/or (c) eliminating a clinical office visit for device removal, which can be made possible through the use of a biodegradable patency device.

The above Detailed Description includes references to the accompanying drawings, which form a part of the Detailed Description. The drawings show, by way of illustration, specific embodiments in which the present devices, methods and kits can be practiced. These embodiments are also referred to herein as “examples.” All publications, patents, and patent documents referred to in this document are incorporated by reference herein in their entirety, as though individually incorporated by reference. In the event of inconsistent usages between this document and those documents so incorporated by reference, the usage in the incorporated reference(s) should be considered supplementary to that of this document; for irreconcilable inconsistencies, the usage in this document controls.

In this document, the terms “a” or “an” are used, as is common in patent documents, to include one or more than one, independent of any other instances or usages of “at least one” or “one or more.” In this document, the term “or” is used to refer to a nonexclusive or, such that “A or B” includes “A but not B,” “B but not A,” and “A and B,” unless otherwise indicated. In this document, the terms “about” and “approximately” are used to refer to an amount that is nearly, almost, or in the vicinity of being equal to a stated amount. In this document, the phrases “nasal side,” “nasal passageway,” and “sinus side” are meant to refer to portions of the patency devices which, when positioned in situ in a maxillary sinus ostium, are closest to those anatomical tissue structures of a subject. For example, when a portion of a patency device is referred to as the “sinus side,” the portion of the device that would be nearest the interior of the maxillary sinus cavity, when implanted, is intended. In contrast, when reference is made to the “nasal side,” the portion of the patency device that would be nearest the nasal passageway, when implanted, is intended. In this document, the term “subject” is meant to include mammals, such as for human applications and veterinary applications. In this document, the phrase “structured for insertion and placement within a maxillary sinus ostium,” or similar, when used in context with the patency device means that the collective structural, configuration, chemical and/or functional features of the device accommodate and utilize the interior dimensions and properties of the tissue structures associated with a maxillary sinus ostium. For example, the phrase can mean that the outer dimensions (e.g., length, width or diameter) of the patency device accommodate the interior ostium dimensions and properties of the ostium tissue.

In the appended claims, the terms “including” and “in which” are used as the plain-English equivalents of the respective terms “comprising” and “wherein.” Also, in the following claims, the terms “including” and “comprising” are open-ended, that is, a system, assembly, device, article, or process that includes elements in addition to those listed after such a term in a claim are still deemed to fall within the scope of that claim. Moreover, in the following claims, the terms “first,” “second,” and “third,” etc. are used merely as labels, and are not intended to impose numerical requirements on their objects.

The above description is intended to be illustrative, and not restrictive. For example, the above-described examples (or one or more features thereof) can be used in combination with each other. Other embodiments can be used, such as by one of ordinary skill in the art upon reviewing the above description. Also, in the above Detailed Description, various features can be grouped together to streamline the disclosure. This should not be interpreted as intending that an unclaimed disclosed feature is essential to any claim. Rather, inventive subject matter can lie in less than all features of a particular disclosed embodiment. For instance, the devices, methods and kits disclosed herein can provide techniques for treatment of a frontal sinus, ethmoid sinus, or sphenoid sinus, in addition to a maxillary sinus. Thus, the following claims are hereby incorporated into the Detailed Description, with each claim standing on its own as a separate embodiment. The scope of the invention should be determined with reference to the appended claims, along with the full scope of equivalents to which such claims are entitled.

The Abstract is provided to comply with 37 C.F.R. § 1.72(b), to allow the reader to quickly ascertain the nature of the technical disclosure. It is submitted with the understanding that it will not be used to interpret or limit the scope or meaning of the claims.

What is claimed is:

1. A device configured for placement in a sinus ostium, comprising:
a first portion;
a second medial portion including a partially open cross-sectional configuration, the partially open cross-sectional configuration maintaining a track of uncovered cilia within the sinus ostium; and
a third portion including one or more outwardly-biased projections, each of the projections having a first end integrally attached to the second medial portion,
wherein the first portion, the second medial portion and the third portion are configured to urge or maintain a patency of the sinus ostium.

2. The device of claim 1, comprising one or more corrugations or lumens along a lateral surface of the first portion, the second medial portion or the third portion, the one or more corrugations or lumens facilitating drainage flow through the sinus ostium.

3. The device of claim 1, wherein a width of the first portion and a width of the third portion are greater than a width of the second medial portion.

4. The device of claim 1, wherein the first portion includes a U-shaped configuration and the third portion includes at least two outwardly-biased projections.

5. The device of claim 4, wherein the U-shaped configuration includes an outward curvature configured to seat against a middle sinus concha and configured to seat within a nasal passageway.

6. The device of claim 1, wherein the first portion includes a conchal spacing structure.

7. The device of claim 1, wherein the third portion includes an expandable interconnecting structure between a first and a second outwardly-biased projection.

8. The device of claim 1, wherein one or more of the first portion, the second portion or the third portion includes a polymeric material.

9. The device of claim 1, wherein one or more of the first portion, the second portion or the third portion includes a metallic material.

10. The device of claim 1, wherein one or more of the first portion, the second portion or the third portion includes a biodegradable material.

11. The device of claim 1, wherein one or more of the first portion, the second portion or the third portion includes a medication for delivery into the sinus ostium or an associated sinus cavity.

12. A method comprising:
placing a sinus ostium patency device extending from a first portion to a third portion within or near a sinus ostium, including placing one or more flexible projections of the third portion at least partially in a maxillary sinus, extending a second portion, intermediate the first and third portions, through the sinus ostium, and placing the first portion under or near a middle sinus concha; and
urging or maintaining an unobstructed channel or passageway through the sinus ostium without circumferentially compromising ciliary function.

13. The method of claim 12, comprising releasing one or more pharmaceuticals or medications into the maxillary sinus or sinus ostium using the sinus ostium patency device.

14. The method of claim 12, wherein placing the sinus ostium patency device includes orienting an asymmetric device body relative to a geometry of the ostium.

15. The method of claim 12, wherein placing the sinus ostium patency device includes moving two flexible projections from a collapsed position to an outwardly-biased position, partially flared against a wall of the sinus ostium.

16. The method of claim 12, comprising reducing an ostium obstruction associated with a concha located adjacent the ostium, including using an extension structure of the sinus ostium patency device to space the concha from the ostium.

17. The method of claim 12, wherein urging or maintaining the unobstructed channel or passageway includes allowing the sinus ostium patency device to self-deploy.

18. The method of claim 12, wherein urging or maintaining the unobstructed channel or passageway includes using an expandable structure to outwardly-bias the sinus ostium patency device.

19. A kit comprising:
the device according to claim 1; and
a set of instructions for using the device to urge or maintain the patency of an ostium associated with a maxillary sinus, a frontal sinus or an ethmoid sinus, without circumferentially compromising ciliary function within the ostium.

20. The kit of claim 19, further comprising a treatment and placement device including a catheter having a side portal and a probe, the probe doubled-backed within the catheter and configured to partially exit via the side portal.
